SEQUENCE LISTING (2 sequences)

SEQ ID NO: 1 - protein, 423 aa, Homo sapiens
  REGION 1..423: note = MISC_FEATURE - GHRH receptor
MDRRMWGAHVFCVLSPLPTVLGHMHPECDFITQLREDESACLQAAEEMPNTTLGCPATWDGLLCWPTAGSGEWVTLPCPDFFSHFSSESGAVKRDCTITGWSEPFPPYPVACPVPLELLAEEESYFSTVKIIYTVGHSISIVALFVAITILVALRRLHCPRNYVHTQLFTTFILKAGAVFLKDAALFHSDDTDHCSFSTVLCKVSVAASHFATMTNFSWLLAEAVYLNCLLASTSPSSRRAFWWLVLAGWGLPVLFTGTWVSCKLAFEDIACWDLDDTSPYWWIIKGPIVLSVGVNFGLFLNIIRILVRKLEPAQGSLHTQSQYWRLSKSTLFLIPLFGIHYIIFNFLPDNAGLGIRLPLELGLGSFQGFIVAILYCFLNQEVRTEISRKWHGHDPELLPAWRTRAKWTTPSRSAAKVLTSMC

SEQ ID NO: 2 - protein, 108 aa, Homo sapiens
MPLWVFFFVILTLSNSSHCSPPPPLTLRMRRYADAIFTNSYRKVLGQLSARKLLQDIMSRQQGESNQERGARARLGRQVDSMWAEQKQMELESILVALLQKHSRNSQG

(19) United States
(12) Patent Application Publication
(10) Pub. No.: US 2025/0073277 A1
(43) Pub. Date: Mar. 6, 2025

(54) METHODS FOR OBTAINING CARDIOMYOGENIC PRECURSOR CELLS

(71) Applicant: University of Miami, Miami, FL (US)

(72) Inventors: Joshua M. HARE, Miami Beach, FL (US); Konstantinos CHATZISTERGOS, Miami, FL (US)

(73) Assignee: University of Miami, Miami, FL (US)

(21) Appl. No.: 18/950,263

(22) Filed: Nov. 18, 2024

Related U.S. Application Data
(62) Division of application No. 16/649,522, filed on Mar. 20, 2020, now Pat. No. 12,168,028.
(60) Provisional application No. 62/562,150, filed on Sep. 22, 2017.

(51) Int. Cl. A61K 35/34; A61K 38/25; C12N 1/38; C12N 5/074; C12N 5/077; C12N 5/10

(52) U.S. Cl. CPC A61K 35/34; A61K 38/25; C12N 1/38; C12N 5/0657; C12N 5/0696; C12N 5/10; C12N 2501/415; C12N 2501/60; C12N 2501/85; C12N 2506/45

(57) ABSTRACT

The disclosure provides a method of obtaining a population of cardiomyogenic precursor cells. The method comprises (a) differentiating induced pluripotent stem cells (iPSCs) to iPSC-derived cardiac precursor cells; and (b) isolating cardiac precursor cells expressing mammalian growth hormone-releasing hormone receptor (GHRHR) to obtain a population of iPSC-derived cardiomyogenic precursor cells.

### CROSS-REFERENCE TO RELATED APPLICATIONS

The present application is a divisional of U.S. patent application Ser. No. 16/649,522, filed on Mar. 20, 2020, which is the United States national stage under 35 U.S.C. § 371 of PCT International Application No. PCT/US2018/052215, filed on Sep. 21, 2018, which claims priority to U.S. Provisional Patent Application No. 62/562,150, filed on Sep. 22, 2017. The entire contents of U.S. patent application Ser. No. 16/649,522, PCT International Application No. PCT/US2018/052215 and U.S. Provisional Patent Application No. 62/562,150 are hereby incorporated by reference in their entirety.

### STATEMENT OF U.S. GOVERNMENTAL INTEREST

This invention was made with government support under grant numbers HL107110 HL084275, HL094848, and HL113460 awarded by the National Institutes of Health (NIH) and National Heart, Lung, and Blood Institute (NHLBI). The government has certain rights in this invention.

### REFERENCE TO AN ELECTRONIC SEQUENCE LISTING

The contents of the electronic sequence listing (51287A-DIV-ST26.xml; Size: 3,471 bytes; and Date of Creation of .xml file from parent .txt file: Nov. 8, 2024) are herein incorporated by reference in their entirety.

### FIELD OF THE INVENTION

The disclosure relates to methods of obtaining cardiomyogenic precursor cells.

### BACKGROUND

Heart disease accounts for 1 in 3 deaths worldwide, largely due to the limited ability of adults to regenerate heart muscle cells in response to damage. Current medical solutions involve surgical intervention and/or lifelong disease management with pharmacologic agents, such as f3-blockers, ACE inhibitors and statins. Novel therapeutic approaches are needed to improve clinical outcomes, reduce treatment duration and limit cost of cardiovascular disease. A promising medical strategy involves induced pluripotent stem cell (iPSC)-based cell therapeutics. iPSCs are a novel regenerative medical tool derived from adult cells through genetic reprogramming, which does not require embryo destruction. Use of iPSCs in clinical practice is hampered by a number of challenges. The development of clinical-grade iPSC-based products requires prior manufacturing of iPSCs from genomic and karyotype stable somatic cells, as well as burdensome steps to minimize the risk of tumorigenicity. There remains a need for an efficient method of generating stem cell-based compositions suitable, e.g., for therapeutic applications.

### SUMMARY

The disclosure is directed to a method of obtaining cardiomyogenic precursor cells. The method comprises differentiating induced pluripotent stem cells (iPSCs) to cardiac precursor cells (e.g., culturing the iPSCs in differentiation medium for a time period of up to 7 days), and isolating cardiac precursor cells expressing mammalian growth hormone-releasing hormone receptor (GHRHR) to obtain a population of cardiomyogenic precursor cells. In various aspects, the isolation step is performed using flow-cytometry or magnetic cell-sorting.

In various aspects, the method further comprises, prior to the differentiation step, culturing a population of iPSCs to expand the iPSC population. Optionally, the differentiation step comprises treating the iPSCs with a glycogen synthase kinase 3 (GSK3) inhibitor, a Wnt inhibitor, somatostatin and recombinant growth hormone releasing hormone (or synthetic analogs of any of the foregoing).

In various embodiments, the method further comprises selecting for c-Kit negative cardiac precursor cells. In some aspects, the isolated iPSC-derived cardiomyogenic precursor cells express nuclear homeobox transcription factors, NK2 Homeobox 5 (Nkx2.5) and Is'1.

The disclosure also provides a population of iPSC-derived cardiomyogenic precursor cells produced by the method described herein. The disclosure further provides a method of treating a cardiac disorder, the method comprising administering to a subject in need thereof the population of iPSC-derived cardiomyogenic precursor cells. The cardiac disorder is, for example, myocardial infarction, angina, congenital heart disease, ischemic or non-ischemic heart failure, heart failure with or without preserved ejection fraction, or dilated cardiomyopathy.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

FIGS. 1A-1E depict temporal gene-expression analyses in an iPSC model of human cardiomyogenesis, illustrating that growth hormone-releasing hormone (GHRH) and growth hormone-releasing hormone receptor (GHRHR) are specifically induced on day 7, following commitment of Brachyury⁺ precardiac cells into NKX2.5 and ISL1 cardioblasts.

FIGS. 2A-2C are representative examples on the assessment of anti-fibrotic effects of cell therapy in pigs with myocardial infarction, using cardiac MRI.

### DETAILED DESCRIPTION

The disclosure relates to methods of obtaining cardiomyogenic precursor cells from iPSCs. This disclosure is the first to utilize the cell-surface molecule, growth hormone-releasing hormone receptor (GHRHR), to isolate cardiomyogenic precursor cells from iPSCs and other cardiac precursor cells. Previous studies utilized adult cardiac stem cells (Florea et al., Circulation Research. 2014; 115:A244; Shiba et al., Curr Pharm Des. 2009; 15(24):2791-2806) which express c-Kit, a marker of cardiac neural crest (CNC) cells but not cardiac mesoderm progenitors (Hatzistergos et al., Proc Natl Acad Sci USA 2015; 112(42):13051-6). In contrast, the method of the disclosure allows isolation of cardiomyogenic cardiac precursor cells, which represent a distinct, immature cell population having the ability to develop into cardiomyocytes. In various embodiments, the isolated iPSC-derived cardiomyogenic precursor cells are c-Kit negative. The method of the disclosure allows collection of potentially unlimited quantities of cardiomyogenic precursor cells.

In one aspect, the disclosure provides a method of obtaining cardiomyogenic precursor cells from iPSCs. The method comprises (a) differentiating iPSCs to cardiac precursor cells. The iPSCs may be human (hiPSCs) or porcine (piPSCs) or any other mammalian species in origin. Cardiac precursor cells are stem cells with the potential to differentiate into multiple cardiac cell types. Cardiomyogenic precursor cells are a subset of cardiac precursor cells with the ability to differentiate into a population of beating cardiomyocytes. Methods of differentiating iPSCs to cardiac precursor cells are known in the art. In various aspects of the method, the iPCSs are treated with a glycogen synthase kinase 3 (GSK3) inhibitor, a Wnt inhibitor, somatostatin and recombinant growth hormone releasing hormone (or synthetic analogs of any of the foregoing) to differentiate iPCSs to cardiac precursor cells. Optionally, the iPSCs are cultured for a time period of up to 7 days under conditions that promote differentiation of the iPSCs into cardiac precursor cells (e.g., 1, 2, 3, 4, 5, 6, or 7 days). Also optionally, the method comprises culturing a population of iPSCs to expand the iPSC population prior to step (a).

The method further comprises (b) isolating cardiac precursor cells expressing GHRHR to obtain a population of iPSC-derived cardiomyogenic precursor cells. Growth hormone-releasing hormone (GHRH) is a highly versatile signaling molecule that exerts a broad range of regulatory influences in the cardiovascular system. The receptor for GHRH, GHRHR, is a G-protein-coupled receptor. The amino acid sequence of human GHRHR is provided as SEQ ID NO: 1. In various aspects, cells expressing GHRHR are separated by undesired cells (i.e., cardiac precursor cells not expressing GHRHR) using flow-cytometry or magnetic cell-sorting.

In various embodiments, the method further comprises a negative selection step to isolate cells which do not express the marker c-kit. Put another way, c-kit-positive cells are optionally removed from the cell population in step (b) to obtain a population of cells that are c-kit-negative. In various aspects, c-kit cardiomyogenic precursor cells are isolated using flow-cytometry, or magnetic cell-sorting is employed to remove c-kit⁺ cells. In some aspects, the resulting cell population from step (b) expresses nuclear homeobox transcription factors, Nkx2.5 and Isl1. Nkx2.5 is a master regulator gene of the cardiac lineage (Behrens et al., Stem Cells Dev. 2013; 22(15): 2211-2220). In various aspects, step (b) further comprises screening cells for expression of Nkx2.5 and Isl1 to obtain a population of cells that express Nkx2.5 and Isl1 in combination with GHRHR.

In further embodiments, the disclosure provides a population of iPSC-derived cardiomyogenic precursor cells isolated using the method described herein. Also provided is a method of treating a cardiac disorder (which may be congenital and/or adult) comprising administering the population of iPSC-derived cardiomyogenic precursor cells to a mammalian subject (e.g., a human subject) in need thereof. In related embodiments, the cardiac disorder is, for example, congenital heart disease, angina, myocardial infarction, ischemic or non-ischemic heart failure, heart failure with or without preserved ejection fraction, or dilated cardiomyopathy.

The population of iPSC-derived cardiomyogenic precursor cells may be

administered via any suitable route of administration which results in delivery to cardiac tissue. For example. in various embodiments, the population of iPSC-derived cardiomyogenic precursor cells are surgically implanted in cardiac tissue. Alternatively, the route of administration is catheter-based, for example, intravenous infusion or intracoronary infusion. In various aspects, the population of iPSC-derived cardiomyogenic precursor cells is delivered via intramyocardial injection. Optionally, the population of iPSC-derived cardiomyogenic precursor cells is administered (e.g., infused or injected) or applied topically to cardiac tissue during surgery. The aforementioned routes of administration are exemplary and not meant to be limiting; other routes of administration are also contemplated (e.g., systemic, parenteral, or intravenous) optionally in connection with implants, reservoirs, or slow release formulations.

The iPSC-derived cardiomyogenic precursor cell population is, in various aspects, autologous or allogeneic to the recipient. The amount of iPSC-derived cardiomyogenic precursor cell administered is that which achieves a desired effect, i.e., to ameliorate, prevent or improve an unwanted condition, disorder, or symptom of the subject, in an appropriate time frame. The population (or dose) of iPSC-derived cardiomyogenic precursor cells comprises, for example, 10×10¹−10×10⁹ cells, delivered in a single administration or multiple administrations per dose. In related embodiments, the iPSC-derived cardiomyogenic precursor cell population is provided as a stand-alone treatment. Alternatively, the iPSC-derived cardiomyogenic precursor cell population is administered as part of a treatment regimen which also includes, for instance, standard-of-care therapies or other cell-, drug-and gene-based therapies.

The disclosure provides a physiologically acceptable (e.g., pharmaceutically acceptable) composition comprising a population of iPSC-derived cardiomyogenic precursor cells and physiologically acceptable excipient(s). Compositions suitable for administration typically include sterile aqueous solutions or dispersions and lyophilized compositions for reconstitution into sterile injectable solutions or dispersions. Typical excipients, diluents, additives, or carriers include, but are not limited to, sterile water, physiological saline or other salt solutions, sugars such as glucose, wetting or emulsifying agents, pH buffering agents, viscosity modifiers, preservatives, and the like.

In one aspect, the method further comprises administering a GHRHR agonist with the population iPSC-derived cardiomyogenic precursor cells. The GHRHR agonist may be present in same composition as the population of cells or may be administered in a separate composition. If administered in a separate composition, the GHRHR agonist may be administered shortly before administration of the population of cells (e.g., within 6 hours, within 4 hours, within 2 hours, within 1 hour, within 30 minutes, within 15 minutes, or within 5 minutes of administration of the population of cells), substantially simultaneous with the administration of the population of cells, or shortly after the population of cells (e.g., within 6 hours, within 4 hours, within 2 hours, within 1 hour, within 30 minutes, within 15 minutes, or within 5 minutes of administration of the population of cells). Alternatively, the GHRHR agonist may be administered after (or before) a longer period of time with respect to the population of cells (e.g., 12 14, or 48 hours before/after administration of the population of cells). Examples of GHRHR agonists include, but are not limited to, CJC-1295, dumorelin, somatorelin, rismorelin, sermorelin, tesamorelin, or GHRH-A (JI-38), or combinations thereof.

In one aspect, the population of iPSC-derived cardiomyogenic precursor cells are modified to increase GHRHR or GHRH protein levels. The sequence of GHRH is provided as SEQ ID NO: 2. Introduction of nucleic acid into host cells to produce a target protein, e.g., GHRHR or GHRH, is well understood in the art. Exogenous nucleic acid may be introduced into host cells via, e.g., expression vector (e.g., viral or non-viral vectors), non-vector based methods (e.g., using naked DNA or DNA complexes), or a combination thereof. Suitable viral vectors include, but are not limited to, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. Other means of facilitating nucleic acid delivery to host cells include, e.g., yeast systems, particle bombardment, nanoparticle-mediated uptake, liposomes, microvesicles, and hydrodynamic-based gene delivery.

The disclosure contemplates both therapeutic and/or prophylactic treatment, as appropriate. It will be appreciated that “treating a cardiac disorder” does not require complete amelioration of the disorder; any improvement in a symptom or manifestation of the disorder confers a beneficial effect on the subject. Methods for measuring cardiac function (e.g., contractile function) are known in the art and are described, for example, in the Textbook of Medical Physiology, Tenth edition, (Guyton et al., W.B. Saunders Co., 2000). For example, cardiac ejection can be monitored using, e.g., echocardiography, nuclear or radiocontrast ventriculography, or magnetic resonance imaging. Other measures of cardiac function include, but are not limited to, myocardial contractility, resting stroke volume, resting heart rate, resting cardiac index, Doppler imaging, cardiovascular performance during stress/exercise. Optionally, cardiac function is increased by at least 5%, 10%, 15%, 20%, 25%, 30%, 40%, 50%, 60%, 70%, 80%, 90%, or even 100% relative to the cardiac function prior to treatment.

In jurisdictions that forbid the patenting of methods that are practiced on the human body, the meaning of “administering” of a composition to a human subject may be restricted to prescribing a controlled substance that a human subject can self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### EXAMPLES

### Example 1: GHRHR is universally expressed by NKX2.5/ISL1⁺ cardiac precursor cells and cardiomyocytes of both mesoderm and CNC origin.

Temporal analysis of GHRHR expression was performed in an in-vitro model of human cardiogenesis using induced hiPSCs (hiPSCs) and SOX10::GFP embryonic stem cells (hESCs); and mouse (in-vivo) cardiogenesis in wild-type (WT), MEF2c-AHF-Cre, Wnt1-Cre2 and ckit-CreERT2/+4-reporter mice.

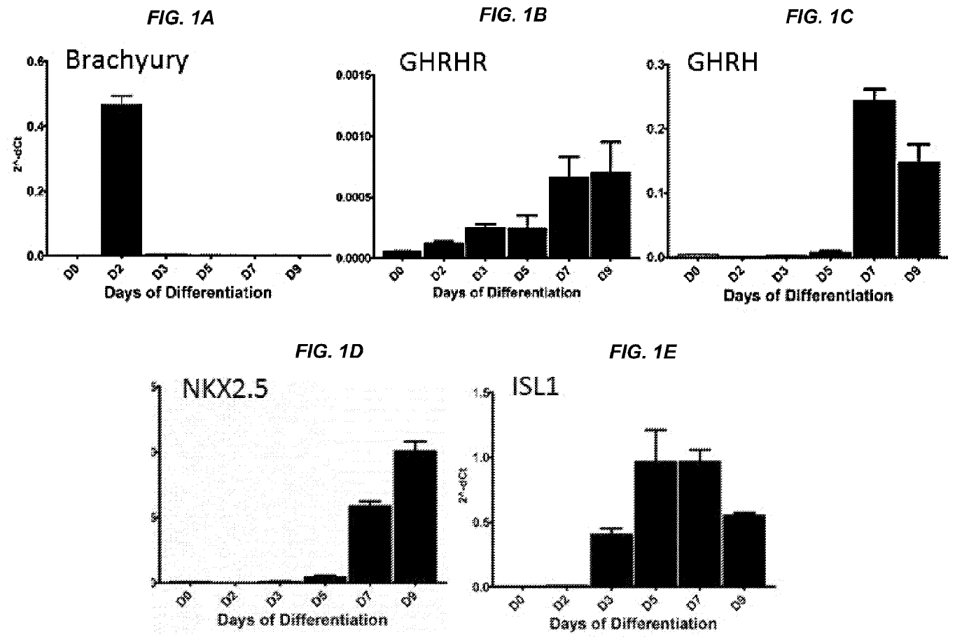

Gene expression and confocal immunofluorescence analyses during chemically-defined, stage-specific, cardiac lineage differentiation indicated that GHRHR is not expressed in undifferentiated hiPSCs or during specification into primitive streak-like Brachyury+ or Mesp1 precardiac cells; but is induced in cardiogenic mesoderm-like cells, at the stage of commitment into NKX2.5⁺ and/or ISL1⁺ CPCs (p=0.001) and persists in Troponin-T⁺ cardiomyocytes. Similarly, experiments modeling cardiac neural crest (CNC) with SOX10::GFP hESCs indicated that GHRHR is not expressed by GFP+ CNCs but is induced following differentiation into NKX2.5⁺ and/or ISL1⁺ derivatives. Importantly, stimulation with 1 μM recombinant GHRH during days 5-7 of hiPSCs differentiation increased NKX2.5 expression 2.5-fold, an effect that was abolished by exposure to 1 μM Somatostatin, a GHRH antagonist (p=0.0009). FIGS. 1A-1E show temporal gene-expression analyses in an iPSC model of human cardiomyogenesis, illustrating that GHRH and GHRHR are specifically induced on day 7, following commitment of Brachyury⁺ precardiac cells into NKX2.5 and ISL1 cardioblasts. Last, in vivo analyses in WT, MEF2c-AHF-Cre, Wnt1-Cre2 and cKit-CreERT2/+ reporter embryonic and postnatal hearts corroborated that GHRHR specifically marks NKX2.5⁺ mesoderm-and CNC-lineage descendants in vivo, whereas GHRHR is not expressed by Wnt1-Cre2 and cKit-CreERT2/+ CNCs descendants that are NKX2.5⁻(NKX2.5-negative).

Together these findings indicate that GHRHR is expressed by NKX2.5⁺ISL1⁺ cardiac precursor cells and cardiomyocytes of both mesoderm and CNC origin.

### Example 2: Derivation of GHRHR+ Cardiomyoblasts from PSCs

To generate GHRHR+ cardiomyoblasts, feeder-free human pluripotent stem cells (hPSCs) were grown in monolayers. At 60-90% confluency (Day 0), the PSC medium was changed to RPM11640 (Thermo Scientific), supplemented with B-27 minus insulin (Thermo Scientific) and 6 pM of the GSK3 inhibitor Chir99021 (Tocris). On day 2, medium was changed to RPMI with B-27 minus insulin (M1). On day 3, M1 medium was supplemented with 5 pM of the Wnt inhibitor IWP2 or IWR1, for 48 h. On day 5, and every other day thereafter, the medium was replenished with fresh M1. On days 8-10, selection of GHRHR+cardiomyoblasts was performed as follows. Differentiated PSC cultures are pretreated for 1 hour with 10 pM of the RHO/ROCK inhibitor Y-27632, dissolved in M1. Cells were rinsed with PBS and dissociated into single-cell suspension with trypLE select. Enrichment of GHRHR+ cells was performed via flow-cytometry or magnetic cell-sorting using a GHRHR antibody (Abcam ab28692). The positive fraction was resuspended in M1 and cryopreserved.

### Example 3: Use of iPSC-derived GHRHR+ CPCs for the Treatment of Adult and Congenital Heart Diseases

The method described herein re-derives highly pure, clinical-grade Nkx2.5+/Isl1+ cardiomyocyte progenitor cells from iPSCs. The method minimizes oncologic and arrhythmogenic risks associated with use of stem cell therapy, because the iPSCs can be derived from clinically safe MSCs, and iPSC—derived cardiomyocyte progenitor cells are selected from unwanted iPSC derivatives, optionally—24 hours prior to their terminal differentiation into beating cardiomyocytes, with a novel, antibody-based selection step for GHRHR.

Stem cells are injected into a subject requiring cardiac intervention in a number of ways including, but not limited to, intramuscular (trans-catheter, direct injection), intracoronary (with or without using the stop-flow technique), intravenous, intramyocardial, surgical, catheter, epicardial placement, and retrograde intracoronary sinus administration. However, despite the low engraftment rate, direct intramyocardial injection with cardiac mapping is a preferred method, as it is directed at the border zone of the scar and it receives the highest concentration of cells in a targeted location.

Mortality status-post MI remains high and those who progress to HF have a 20-30 percent chance of death within the year despite standard pharmacological therapy. In combination with standard medical therapy, stem cell therapy will aid in the healing process by salvaging ischemic tissue in the subacute setting, thus reducing the proarrhythmic focus and subsequent mortality rate. The inflammatory milieu after an MI is hostile to native stem cells and cardiomyocytes alike. iPSCs can be introduced at, e.g., two weeks after the event, at which time the autologous cells will have had time to replicate and the inflammation will have begun subsiding. Subjects with congenital heart diseases, chronic ischemic HF, HF with preserved ejection fraction and dilated cardiomyopathy are contemplated as recipients of the population of I PSC-derived cardomyocyte progenitor cells of the disclosure.

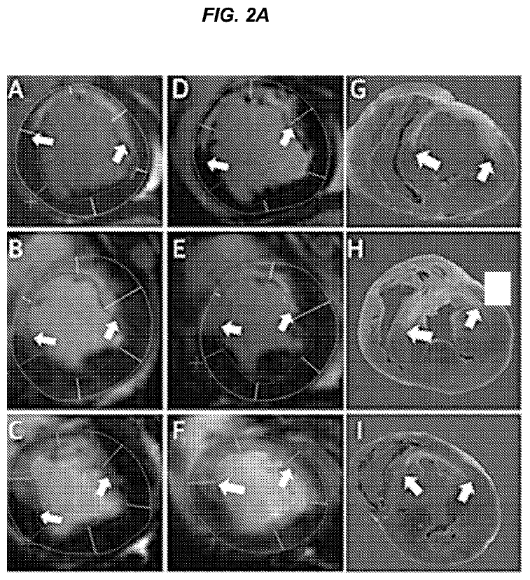

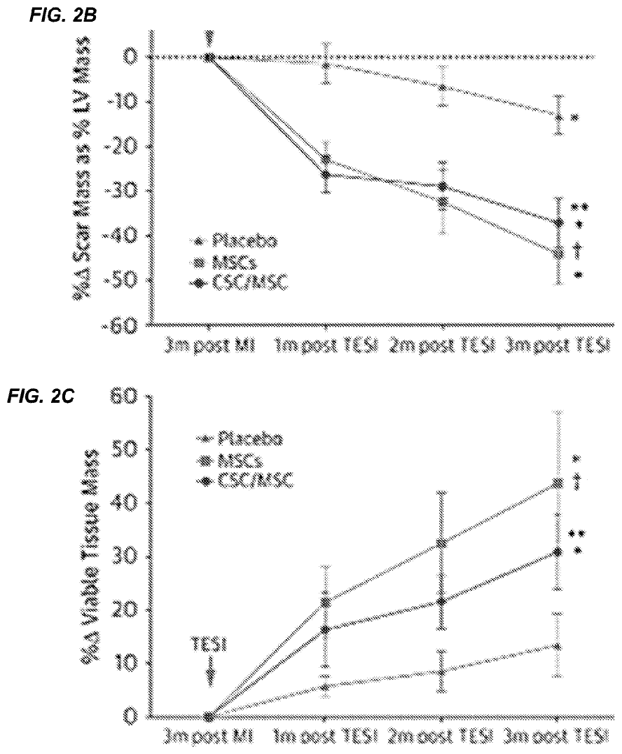

A head-to-head placebo-controlled comparison of GHRHR-sorted porcine iPSCs (piPSC)-derived cardiac progenitor cells with their parent porcine MSC line and unsorted piPSCs (Day 7 of cardiac differentiation) in a sex-mismatched porcine model of cell therapy in response to (sub) acute myocardial infarction (MI), will be performed. FIG. 2 shows examples of a recent study, in which this animal model was utilized to test the safety and efficacy of a combination cell therapy, with adult cardiac stem cells and mesenchymal stem cells (MSCs) (Karantalis et al., J Am Coll Cardiol. 2015; 66(18):1990-9). The results from this study led to IND approval for the CONCERT-HF clinical trial (ClinicalTrials.gov Identifier: NCT02501811).

The goal of the study is to confirm the safety and efficacy of the use of iPSC-derived GHRHR+ cardiomyocyte progenitor cells in the treatment of adult and congenital heart diseases. The porcine model of experimental MI will be employed to perform a sex-mismatched, placebo-controlled, double-blinded, dose-response comparison of porcine iPSC-derived GHRHR+ cardiomyocyte progenitor cells, with their parent MSC product and iPSC-derived cells without GHRHR sorting. Treatment will be delivered in a blinded fashion, via transendocardial stem cell injection (TESI) as previously described (Karantalis et al., J Am Coll Cardiol. 2015; 66(18):1990-9). Infarcted animals will be randomized to receive 5, 25 or 100×10⁶ male iPSC-derived cardiomyocyte progenitor cells, 100×10⁶ male MSCs from which the iPSC-cardiomyocyte progenitor cells were derived, 100×10⁶ unsorted male piPSCs derivatives or placebo. Animals will be followed for 3 months prior to euthanasia. A complete necropsy will be performed and tissues inspected for ectopic tissue formation by a trained veterinary pathologist.

Populations of 6.5×10⁸ cGLP-grade GHRHR+ cardiomyocyte progenitor cells and 5×10⁸ cGLP-grade iPSC derivatives not sorted for GHRHR will be produced. The two iPSC-based cell products will be derived from a single male, porcine iPSC line, which were generated from MSCs under feeder-free, virus-free, transgene-free, chemically defined conditions. In addition, cryopreserved 1×10⁹ cells from the same MSC line will be used as the current standard in cell-based therapy for heart disease, in head-to-head comparison with their iPSC derivatives.

The procurement of 30 female Yorkshire swine and induction of experimental myocardial infarction will be performed. Animals will undergo closed-chest ischemic reperfusion myocardial infarction (MI), in which MI is induced by inflation of a coronary angioplasty balloon in the mid left anterior descending (LAD) artery for 90 minutes as previously described (Karantalis et al, J Am Coll Cardiol. 2015; 66(18):1990-9). The following safety endpoints will be recorded before and after MI: survival; body weights; continuous cardiac rhythm monitoring for ventricular or supraventricular arrhythmias using implanted monitoring devices (“REVEAL DX 9528” and “REVEAL XT 9529” [Medtronic, Minneapolis, MN]); circulating blood markers (hematology, chemistry, and markers of cardiac injury CPK, CKMB, and Troponin I). Heart function is assessed with cardiac MRI using a 3.0T clinical MR scanner (Magnetom, Siemens).

Randomized, double-masked, placebo-controlled, preclinical studies also will be performed. This study entails the randomization of the MI animals in the following 6 TESI groups (5 animals/group): 5, 25 or 100×10⁶ male iPSC-derived CPCs, 100×10⁶ male MSCs from which the iPSC-CPCs were derived, 100×10⁶ unsorted male piPSCs derivatives, or placebo (plasmalyte). TESI is performed with the NOGA myostar catheter under the NOGA electromechanical mapping system. Animals are followed for 3 months following therapy for safety and efficacy endpoints.

To assess safety and efficacy endpoints, animals are euthanized 3 months after TESI, and safety and efficacy endpoints are analyzed in a blinded manner. In addition, gross and microscopic tissue analysis from brain, liver, spleen, kidney, lung and ileum for the presence of neoplastic tissue will be performed at the end of the study. Furthermore, the engraftment of allografts will be evaluated based on the detection of Y-chromosome with Fluorescent In Situ Hybridization as previously described (Karantalis et al, J Am Coll Cardiol. 2015; 66(18):1990-9).

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed:

1. A method of obtaining a population of cardiomyogenic precursor cells, the method comprising:
(a) differentiating induced pluripotent stem cells (iPSCs) to iPSC-derived cardiac precursor cells; and
(b) isolating cardiac precursor cells expressing mammalian growth hormone-releasing hormone receptor (GHRHR) to obtain a population of iPSC-derived cardiomyogenic precursor cells.

2. The method of claim 1, further comprising, prior to step (a), culturing a population of iPSCs to expand the iPSC population.

3. The method of claim 1, further comprising, selecting for c-Kit negative cardiac precursor cells.

4. The method of claim 1, wherein step (a) comprises treating iPSCs with a glycogen synthase kinase 3 (GSK3) inhibitor, a Wnt inhibitor, somatostatin and recombinant growth hormone releasing hormone or their synthetic analogs.

5. The method of claim 1, wherein step (a) is performed for a time period of up to 7 days.

6. The method of claim 1, wherein step (b) is performed using flow-cytometry or magnetic cell-sorting.

7. The method of claim 1, wherein the isolated iPSC-derived cardiomyogenic precursor cells express nuclear homeobox transcription factors, Nkx2.5 and Isl1.

8. A population of iPSC-derived cardiomyogenic precursor cells produced by the method of claim 1.

9. A population of iPSC-derived cardiomyogenic precursor cells produced by the method of claim 2.

10. A method of treating a cardiac disorder, the method comprising administering to a subject in need thereof the population of iPSC-derived cardiomyogenic precursor cells of claim 8.

11. The method of claim 10, wherein the cardiac disorder is myocardial infarction, angina, congenital heart disease, ischemic or non-ischemic heart failure, heart failure with or without preserved ejection fraction, or dilated cardiomyopathy.

12. A method of treating a cardiac disorder, the method comprising administering to a subject in need thereof the population of iPSC-derived cardiomyogenic precursor cells of claim 9.

13. The method of claim 10, wherein the population of iPSC-derived cardiomyogenic precursor cells are administered via surgical implantation, intravenous infusion, intracoronary infusion, or intramyocardial injection.

14. The method of any claim 10, wherein the population of iPSC-derived cardiomyogenic precursor cells administered to the subject comprises between 10×10¹-10×10⁹ cells.

15. The method of claim 10, further comprising administering to the subject a GHRHR agonist.

16. The method of claim 15, wherein the GHRHR agonist is CJC-1295, dumorelin, somatorelin, rismorelin, sermorelin, tesamorelin, or GHRH-A (JI-38), or a combination thereof.

17. The method of claim 10, wherein the population of iPSC-derived cardiomyogenic precursor cells are modified to increase GHRHR protein levels.

18. The method of claim 10, wherein the population of iPSC-derived cardiomyogenic precursor cells are modified to increase growth hormone-releasing hormone (GHRH) protein levels.
